(19) United States
(12) Patent Application Publication
(10) Pub. No.: US 2009/0012516 A1
(43) Pub. Date: Jan. 8, 2009

(54) Electrosurgical system

(75) Inventors: Richard J. Curtis, Newport (GB); Michael David Newton, Newport (GB)

Correspondence Address: NIXON & VANDERHYE, PC, 901 NORTH GLEBE ROAD, 11TH FLOOR, ARLINGTON, VA, 22203, US

(73) Assignee: Gyrus Medical Limited, Cardiff (GB)

(21) Appl. No.: 12/213,298

(22) Filed: Jun. 17, 2008

(30) Foreign Application Priority Data
Jun. 19, 2007 (GB) 0711868.0

(51) Int. Cl. A61B 18/04

(52) U.S. Cl. 606/34

(57) ABSTRACT

An electrosurgical system includes an electrosurgical generator (1) with a plurality of output connections (2, 3, 4), only one of the output connections at any one time being active in that it is able to receive radio frequency power. A plurality of electrosurgical assemblies, each including an electrosurgical instrument (5, 6, 7) and a cable (8, 9, 10) are connected to the generator (1) via the output connections. The electrosurgical assemblies each include indication means such as lamps (17, 18, 19), illuminating when that particular electrosurgical assembly is connected to the active output connection. A switch means, such as a footswitch (11) or handswitches (24, 27) on the instruments, send a signal to the generator to cause an RF waveform to be provided at the active output connection.

### BACKGROUND OF THE INVENTION

This invention relates to an electrosurgical system including an electrosurgical generator, and a plurality of electrosurgical instruments for use therewith.

The majority of electrosurgical generators are designed to have only one instrument attached to the generator at any one time. However, there is an increasing trend, with ever more sophisticated electrosurgery systems, to have multiple instruments connected to the generator at any one time. U.S. Pat. Nos. 5,342,356 and 6,508,809 are two examples of electrosurgical systems in which multiple instruments can be connected to a single generator. These patents describe how shaped connectors, colour coding and distinct symbols can be used to assist with the connection of these multiple instruments, and to ensure that the correct instrument is connected to the appropriate output of the generator.

However, even when all of these instruments have been connected, a problem still remains. Most of these electrosurgical generators only allow for the operation of one of the attached instruments at any one time, and there is a problem for the user in knowing which of the many instruments is currently selected for operation. It is an aim of the present invention to provide an improved electrosurgical system that alleviates this problem.

### SUMMARY OF THE INVENTION

Accordingly, an electrosurgical system is provided comprising:

i) an electrosurgical generator, comprising:
a) at least one source of radio frequency (RF) power;
b) a plurality of output connections;
c) selection means operable by the user of the electrosurgical system to select which one of the output connections, is able to receive RF power from the source to become an active output connection.
d) a controller adapted to control the supply of RF power from the source to the active output connection;

ii) a plurality of electrosurgical assemblies, each including an electrosurgical instrument and a cable connecting that electrosurgical instrument to one of the output connections, each electrosurgical assembly being connectable to a respective output connection of the generator, the electrosurgical assemblies each including an indication means having first and second states, each indication means changing from its first state to its second state when that particular electrosurgical assembly is connected to the active output connection; and

iii) switch means adapted to send a signal to the controller to cause an RF waveform to be provided at the active output connection;

wherein the indication means of each electrosurgical assembly comprises an area of that electrosurgical assembly capable of being illuminated; and

wherein each electrosurgical instrument includes a distal tip capable of the treatment of tissue, and the area of the associated electrosurgical assembly capable of being illuminated includes the distal tip of the instrument; and

wherein the system is such that illumination from each electrosurgical assembly area is directable on to the tissue to be treated from the distal tip of the associated instrument.

Thus, when an instrument is active, not only is this shown by the illumination of the illuminated area, but the distal tip of that instrument is illuminated to aid visualisation of the target surgical site.

The indication means shows the user of the electrosurgical system which of the various electrosurgical assemblies is “active”, and will be energised when the user activates the switch means. This helps to avoid confusion in the operating theatre, as well as the need for the user to refer back to the generator before activating the switch means.

Illuminated lamps on electrosurgical instruments are known, examples being given in U.S. Pat. Nos. 3,875,945, 4,800,878, 5,817,091 and 6,676,660. However, whereas these lamps may be used to indicate that energy is being delivered to the instrument, none of these systems use the lamps to indicate the one active instrument from a plurality of instruments.

Typically, each said electrosurgical assembly area is located on the associated electrosurgical instrument. The illuminating means is typically an LED, or alternatively an incandescent bulb.

In one convenient arrangement, the system is such that each indication means changes from a non-illuminated condition constituting the first state, to an illuminated condition constituting the second state when the associated electrosurgical assembly is connected to the active output connection. Alternatively, the system may be such that each indication means changes from a first colour constituting the first state, to a second colour constituting the second state when the associated electrosurgical assembly is connected to the active output connection. Thus, all of the electrosurgical instruments correctly connected to the generator would show an indicator with a first colour, but the one instrument currently connected to the active output connection would show with a second colour. When the user changes the active output connection using the selection means (which could typically be by means of a handswitch on the instrument, buttons on the generator, or a menu-driven selection process using a footswitch), the previously-selected instrument would revert to the first colour, and the newly-selected instrument would become “active” and show the second colour. Alternatively, the active instrument could display a flashing illumination, while the non-active instruments could display either a constant illumination, or no illumination.

In a further refinement of the invention, the system is such that each indication means changes to a further condition when the RF waveform is being provided to the associated electrosurgical assembly. Thus, not only is an indication given of which instrument is active, a further indication is given when the active instrument is actually in use. In one arrangement, the further condition comprises a further colour, for example green indicating the active instrument changing to orange when the instrument is actually in use. Alternatively, the further colour can change depending on in which mode an instrument is being used. For example, the illumination can be yellow when an instrument is being used to cut tissue, or blue when it is being used to coagulate tissue. Alternatively, the further condition indicating actual delivery of electrosurgical energy comprises a flashing illumination.

Conceivably, the further condition comprises an illumination that varies depending on the nature of the RF waveform provided to the associated electrosurgical assembly. For example, the illumination could vary either in colour or intensity depending on the RF waveform provided to the associated electrosurgical assembly, one example being different intensities of illumination depending on the power setting selected by the user of the system.

In one convenient arrangement, each electrosurgical instrument includes a handswitch, and the area of the associated electrosurgical assembly capable of being illuminated comprises the handswitch. Where an instrument includes two handswitches (one for each mode of operation), both may be illuminated when the instrument is the active instrument, for example the “cut” handswitch being illuminated in yellow, and the “coag” handswitch being illuminated in blue. Thus, not only should there be no confusion as to which instrument in active, but also no confusion as to which mode of operation is being selected.

Those skilled in the art will appreciate that optical fibres, light guides or other known methods can be used to transfer the illumination to the distal tip of an instrument, and that, as previously described, the colour of the illumination can be chosen or varied to match the status or usage mode of that instrument.

### BRIEF DESCRIPTION OF THE DRAWINGS

The invention will now be described in more detail, by way of example only, with reference to the accompanying drawings, in which;

FIG. 1 is a schematic view of an electrosurgical system constructed in accordance with the invention;

FIG. 2 is a side view of an electrosurgical instrument for use with the system of FIG. 1;

FIG. 3 is a side view of an alternative electrosurgical instrument for use with the system of FIG. 1;

FIG. 4 is a perspective view of the distal tip of an electrosurgical instrument forming part of the system of FIG. 1; and

FIG. 5 is a schematic view of an electrosurgical system similar to that of FIG. 1.

### DETAILED DESCRIPTION OF PREFERRED EMBODIMENT

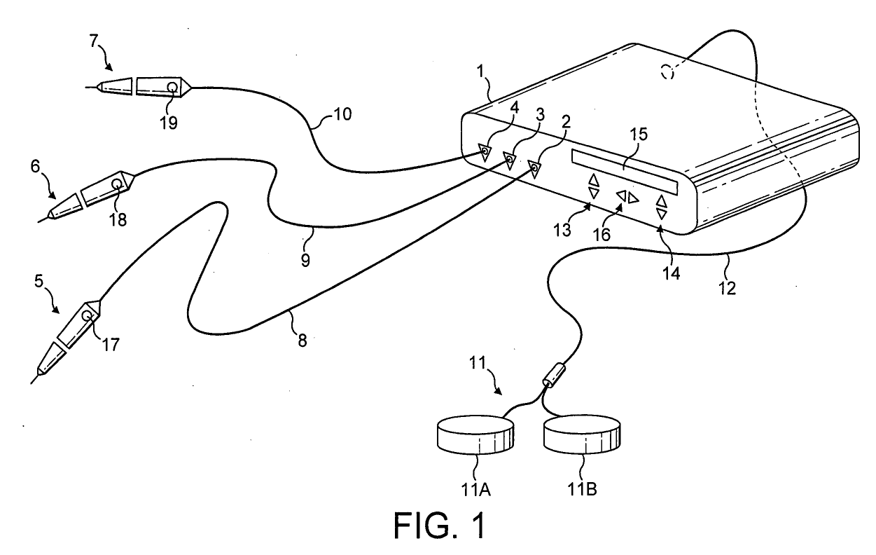

Referring to FIG. 1, a generator 1 has a output sockets 2, 3, 4, providing a radio frequency (RF) output for electrosurgical instruments 5, 6, 7, via respective connection cords 8, 9, 10. Activation of the generator 1 may be performed from the instruments 5, 6, 7, via handswitches (not shown), or by means of a footswitch unit 11, as shown, connected to the rear of the generator by a footswitch connection cord 12. In the illustrated embodiment, the footswitch unit 11 has two footswitches 11A and 11B for selecting a coagulation mode and a cutting mode of the generator 1 respectively. The generator front panel has push buttons 13 and 14 for respectively setting coagulation and cutting power levels, which are indicated in a display 15. Push buttons 16 are provided as a means for selection between the instruments 5, 6, and 7.

The instruments 5, 6, 7, have lamps 17, 18, 19, to show the status of the instruments. If, for example, a user of the electrosurgical system wishes to use the instrument 5, the user presses the push buttons 16 to toggle between the instruments. When the instrument 5 has been selected, the lamp 17 will illuminate on the instrument 5 to show that that instrument is the instrument selected to be “active”. No RF energy will flow to the instrument 5 until the footswitch 11 is depressed, at which time the lamp 17 will flash repeatedly on and off to show that the instrument is in use. When the user wishes to use an alternative instrument, such as the instrument 6, the user presses the push buttons 16 at which point the generator toggles between the three instruments, with each of the lamps 17, 18 and 19 being illuminated in turn. When the lamp 18 is illuminated (showing that the instrument 6 is selected as the active instrument), the user stops pressing the push buttons 16 and is ready to activate the footswitch 11.

If there is a pause in the surgical procedure, or if instruments other than the instruments 5, 6, 7, are used, the surgeon may not be 100% sure which instrument was last used. With the system of FIG. 1, when the surgeon returns to use the instruments 5, 6, 7, it will be immediately apparent from the lamps 17, 18, 19 which of the instruments is selected for use. If the currently-active instrument is not the desired instrument, the user will know that a change has to be made, without having to experiment to find out which instrument is currently active.

The lamps 17, 18, 19, may be simple bulbs, or alternatively LEDs, incandescent or neon lights or electro-luminescent materials. The lamps 17, 18, 19 may have the ability to change colour, to show the different status of each instrument. For example, instead of the simple on/off illumination described previously, the following colour codes could be employed;

No Illumination—instrument not connected to the generator 1;
Orange—instrument connected to the generator 1 but not selected as active instrument;
Green—instrument connected to the generator 1 and selected as the active instrument;
Yellow—instrument delivering a cutting RF signal;
Blue—instrument delivering a coagulating RF signal;
Red—fault condition.

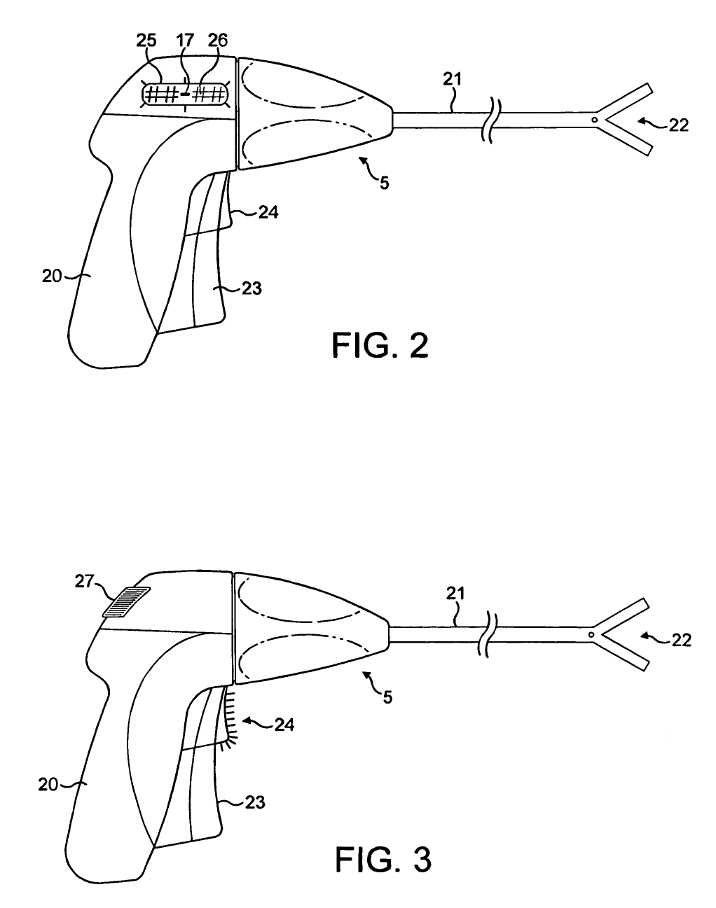

FIG. 2 shows an example of an instrument 5, such as a forceps instrument including a handpiece 20, a shaft 21 and a jawed end-effector 22 at the distal end of the shaft. An actuating handle 23 opens and closes the jaws 22, while a handswitch 24 activates a cutting mechanism (not shown), which may either be the movement of a mechanical blade or the sending of a cutting signal to an electrosurgical cutting element. The lamp 17 is located beneath a transparent panel 25, such that, when the lamp is illuminated, an indication 26 is displayed. The indication 26 may be the name or model number of the instrument, or a company name or logo. As previously described, the lamp 17 may provide a simple on/off illumination, or include different colours or a flashing on/off illumination depending on the circumstances.

FIG. 3 shows a similar forceps instrument 5, but one having a handswitch 27 in addition to the previously-described handswitch 24. When actuation of the handle 23 has closed the jaws 22, activation of the handswitch 24 causes the generator 1 to send a coagulating RF signal to the end-effector 22, while activation of the handswitch 27 causes the generator to send a cutting RF signal to the end-effector. Both the handswitches 24 and 27 are translucent and have a lamp therein, so that, when the instrument 5 is selected as the active instrument, the handswitches 24 and 27 are illuminated. Either the lamps or the translucent handswitch casings are coloured, such that the coagulation handswitch 24 is illuminated with a blue colour, while the cutting handswitch 27 is illuminated with a yellow colour. When the push buttons 16 on the generator 1 are used to toggle to a different instrument, such that the instrument 5 is no longer the active instrument, the lamps are no longer illuminated.

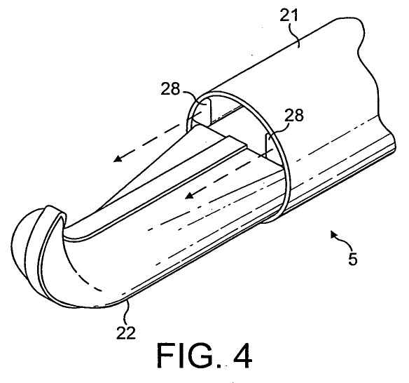

FIG. 4 shows an instrument 5 with an alternative end-effector 22. The shaft 21 includes optical light guides 28, such that, when the instrument 5 is selected as the active instrument, light from the lamp 17 is transmitted to the end-effector 22. Thus, not only does the user of the instrument have a clear indication that this particular instrument is selected as the active instrument, but also an illumination of the distal tip of the instrument and/or the target surgical site.

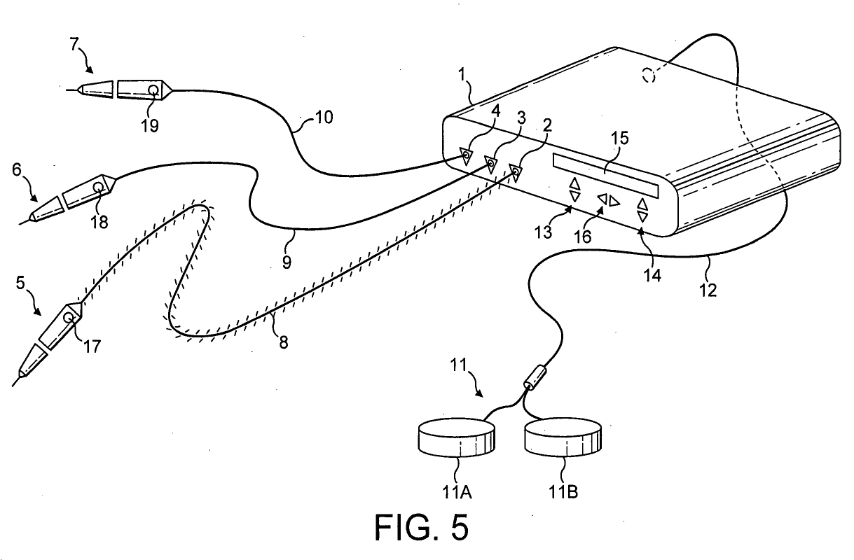

FIG. 5 shows an electrosurgical system similar to that of FIG. 1, but in which each of the connection cords 8, 9, 10 is provided with an electro-luminescent material. When the push buttons 16 are used to toggle between the various instruments 5, 6, 7, a small current is sent to the electro-luminescent material of the active instrument, causing it to become illuminated. FIG. 5 shows the instrument 5 being the active instrument, such that the cord 8 is illuminated. This allows the surgeon to have a clear indication of which instrument is ready to be used. Different casings or electro-luminescent materials can be used, such that the cord for each instruments shows as a different colour when illuminated.

1. An electrosurgical system comprising:
i) an electrosurgical generator, comprising:
a) at least one source of radio frequency (RF) power;
b) a plurality of output connections; c) selection means operable by the user of the electrosurgical system to select which one of the output connections, is able to receive RF power from the source to become an active output connection.
d) a controller adapted to control the supply of RF power from the source to the active output connection;
ii) a plurality of electrosurgical assemblies, each including an electrosurgical instrument and a cable connecting that electrosurgical instrument to one of the output connections, each electrosurgical assembly being connectable to a respective output connection of the generator, the electrosurgical assemblies each including an indication means having first and second states, each indication means changing from its first state to its second state when that particular electrosurgical assembly is connected to the active output connection; and
iii) switch means adapted to send a signal to the controller to cause an RF waveform to be provided at the active output connection;
wherein the indication means of each electrosurgical assembly comprises an area of that electrosurgical assembly capable of being illuminated; and
wherein each electrosurgical instrument includes a distal tip capable of the treatment of tissue, and the area of the associated electrosurgical assembly capable of being illuminated includes the distal tip of the instrument; and
wherein the system is such that illumination from each electrosurgical assembly area is directable on to the tissue to be treated from the distal tip of the associated instrument.

2. An electrosurgical system according to claim 1, wherein the system is such that each indication means changes from a non-illuminated condition constituting the first state, to an illuminated condition constituting the second state when the associated electrosurgical assembly is connected to the active output connection.

3. An electrosurgical system according to claim 1, wherein the system is such that each indication means changes from a first colour constituting the first state, to a second colour constituting the second state when the associated electrosurgical assembly is connected to the active output connection.

4. An electrosurgical system according to claim 1, wherein the system is such that each indication means changes to a second state comprising a flashing illumination.

5. An electrosurgical system according to claim 1, wherein the system is such that each indication means changes to a further condition when the RF waveform is being provided to the associated electrosurgical assembly.

6. An electrosurgical system according to claim 5, wherein the further condition comprises a further colour.

7. An electrosurgical system according to claim 5, wherein the further condition comprises a flashing illumination.

8. An electrosurgical system according to claim 7, wherein the further condition comprises an illumination that varies depending on the nature of the RF waveform provided to the associated electrosurgical assembly.
